(19) United States
(12) Patent Application Publication
(10) Pub. No.: US 2016/0038386 A1
(43) Pub. Date: Feb. 11, 2016

(54) Body Scrub Composition and Method

(71) Applicant: Joey Hoy, Galveston, TX (US)

(72) Inventor: Joey Hoy, Galveston, TX (US)

(21) Appl. No.: 14/887,577

(22) Filed: Oct. 20, 2015

(51) Int. Cl. A61K 8/20; A61Q 19/10; A61K 8/49; A61K 8/60; A61K 8/92

(52) U.S. Cl. CPC A61K 8/20; A61K 8/60; A61K 8/922; A61K 8/498; A61Q 19/10

(57) ABSTRACT

Disclosed is a composition for a face and body scrub and method of producing the same. In one embodiment, the composition includes salt or sugar, emollient, fragrance, vitamin, and color, wherein the emollient includes soybean oil and tee tree oil. The composition is uniform in texture and is suitable for use as an exfoliator while providing antibacterial effects.

### FIELD OF THE INVENTION

The present invention generally relates to cosmetic compositions for exfoliating skin. More particularly, the present invention is directed to a body scrub composition that results in a uniform texture and appearance and that can provide antibacterial effect.

### BACKGROUND OF THE INVENTION

Body and face scrubs offer many therapeutic, mineralization benefits. For example, body and face scrubs increase blood flow and assists in blood circulation. The scrubs also help exfoliate and hydrate skin by removing dead skin cells. For this reason, body and face scrubs are popular body treatments.

Generally, body and face scrubs are stored in jars or containers from which a user can scoop a small amount to apply to the body or face. When the body and face scrubs are stored in jars or containers, the ingredients in the scrubs tend to separate over time; and denser ingredients, such as exfoliants or scrubbing agents, settle at the bottom of the jar or container. Thus, the user must stir the scrub before using it so that the scrub is uniform and the user can apply a proper mixture of the scrub to his or her body and/or face. In this regard, the invention described herein addresses this problem.

### DESCRIPTION OF THE PRIOR ART

Various types of body scrub and cosmetic scrub compositions have been disclosed in the prior art. These include body scrub and scrubs that have been patented and published in patent application publications. The foregoing is a list of references deemed most relevant to the present disclosure, which are herein described for the purposes of highlighting and differentiating the unique aspects of the present invention, and further highlighting the drawbacks existing in the prior art.

The prior art body scrub compositions generally comprise a scrubbing agent, e.g., salt, and emollient. Specifically, Vinski, U.S. Pat. No. 6,551,603, discloses a cosmetic salt scrub product that is relatively uniform in composition. In one embodiment, Vinski discloses a salt scrub composition comprising 20 to 95% by weight of sodium sesquicarbonate and 2 to 20% by weight of an oil. In this regard, the sesquicarbonate and the oil are present in a weight ratio ranging from about 85:15 to about 75:25. In other embodiments, water-soluble polymer and colored wax spheres may be added. While Vinski claims a salt scrub product that is relatively uniform in composition, the ingredients of the composition in Vinski differ from the present invention.

Similarly, Stavroff, U.S. Pat. No. 5,866,145, discloses a composition having Dead Sea salt and emollient, wherein the emollient is silicone oil and is present in about 32.67% by weight. The composition as disclosed in Stavroff, however, differ from the present invention and do not explicitly claim vitamin E. Samelson, U.S. Patent Application Publication Number 2008/0274204, discloses a composition having vitamin E. Samelson, however, does not disclose a scrub composition including salt and other food grade ingredients. Instead, Samelson discloses Dead Sea minerals, wherein the Dead Sea minerals comprise 51% of the composition.

Gitomer, U.S. Pat. No. 7,195,770, discloses a body cleaning composition that includes crushed basalt, salt, and oil, wherein the oil is selected from the following group: peppermint, spearmint, grapefruit, rosemary, orange, lemon, soybean, safflower, and sweet almond. In a preferred embodiment, the salt is present in an amount of 0 to 15% by weight based upon the total weight of the composition. In this regard, Gitomer does not disclose a composition having tea tree oil.

Revivo, U.S. Pat. No. 7,101,578, discloses a composition hat includes 40 to 50% by weight dead sea salt, 28 to 34% by weight safflower seed oil, 11.5 to 14.05% by weight dimethicone, 7.4 to 9.05% by weight silica, 1 to 3% by weight fragrance, 0.14 to 0.16% by weight avocado oil, 0.14 to 0.165 by weight jojoba oil, 0.05 to 0.6% by weight vitamin A, 0.05 to 0.6% by weight vitamin B, 0.05 to 0.6% by weight vitamin C, 0.05 to 0.6% by weight vitamin D, 0.05 to 0.6% by weight vitamin E, and 0.05 to 0.6% by weight vitamin K. Revivo, however, does not disclose food grade ingredients.

Ciesar, PCT Patent Application Publication Number WO 2000/004867, discloses a body polish comprising sea salt, carrier oil, essential oil, and/or fragrance oil, and a lecithin-derived phospholipid emulsifier. Ciesar, however, does not disclose use of food grade ingredients.

Soliman, U.S. Patent Application Publication Number 2005/0158351, discloses anhydrous skin cleanser, which includes 20 to 90% by weight emollient, 0.5 to 15% by weight ionic surfactant, 5 to 40% by weight exfoliative, and 0.5 to 10% by weight oil gelling agent. While Soliman discloses some of the ingredients disclosed in the present invention, the foregoing ingredients disclosed in Soliman are not safe for consumption.

Rau, U.S. Patent Application Publication Number 2006/0104932, also discloses exfoliating skin care product comprising anhydrous liquid and water-soluble particulate material that dissolves in water between 20 to 90 seconds at 40° C. Thus, Rau does not disclose each of the ingredients of the present composition.

In this regard, the intent and purpose of the foregoing prior art references differ significantly from the present invention in that none of the prior art references disclose a face and body scrub that is completely uniform in composition, and that is composed of natural, food grade ingredients. Thus, the compositions in the prior art are limited in that the ingredients tend to separate when stored and that they are not safe if consumed. Additionally, none of the prior art scrub compositions comprise soybean oil, all-natural fragrance, all-natural color, vitamin E or tea tree oil, and non-iodized fine granular salt or sugar.

### SUMMARY OF THE INVENTION

In view of the disadvantages inherent in the known types of compositions for body scrubs now present in the prior art, the present invention provides an improved body scrub that is homogeneous in composition.

The following discloses a simplified summary of the specification in order to provide a basic understanding of some aspects of the specification. This summary is not an extensive overview of the specification. It is intended to neither identify key or critical elements of the specification nor delineate the scope of the specification. Its sole purpose is to disclose some concepts of the specification in a simplified form as to prelude to the more detailed description that is disclosed later.

In one embodiment, the present body scrub comprises 1 gal soybean oil, 4 fl oz fragrance, 2 fl oz color, and 2 fl oz vitamin E or tea tree oil per 100 lbs of non-iodized fine granular salt or sugar. If sugar is used, the body scrub further comprises 1 fl oz polysorbate 80. Preferably, each of the ingredients comprise all natural and food grade compounds so that the composition is safe even if consumed accidentally.

The method of producing the instant body scrub includes the steps of combining all of the liquids in a mixing barrel. Once the liquids are mixed, one half of the salt or sugar is added and combined until the mixture scrapes the sides of the barrel. Thereafter, the remaining half of the salt or sugar is added until the mixture is substantially uniform. The ingredients are combined at approximately 21° C. to 22° C. and the resulting product is kept at even temperature.

It is therefore an object of the present invention to provide a body scrub composition that does not separate or settle when stored for an extended period of time to as to eliminate any need for stirring.

It is another object of the present invention to provide a body scrub position that provides antimicrobial and antibacterial effects, thereby providing a cleaning action for approximately a 12-hour period.

It is still another object of the present invention to provide a body scrub composition that can be used as an exfoliator to remove dead skin cells while preventing skin irritation.

It is still another object of the present invention to provide a body scrub composition that comprises food grade ingredients that does not cause harm to an individual if ingested accidentally.

It is still another object of the present invention to provide a body scrub composition that comprises various fragrance and color.

It is still another object of the present invention to provide a body scrub composition that does not comprise artificial preservatives.

A final object of the present invention to provide a body scrub composition that may be readily fabricated from materials that permit relative economy and commensurate with durability.

In the light of the foregoing, these and other objects are accomplished in accordance of the principles of the present invention, wherein the novelty of the present invention will become apparent from the following detailed description and appended claims.

### DETAILED DESCRIPTION OF THE INVENTION

The present invention is directed towards a composition for skin exfoliators. Various modifications obvious to one skilled in the art are deemed to be within the spirit and scope of the present invention.

The word “exemplary” is used herein to mean serving as an example, instance, or illustration. Any aspect or design described herein as “exemplary” is not necessarily to be construed as preferred or advantageous over other aspects or designs. Rather, use of the word exemplary is intended to disclose concepts in a concrete fashion. As used in this application, the term “or” is intended to mean an inclusive “or” rather than an exclusive “or.” Additionally, the articles “a” and “an” as used in this application and the appended claims should generally be construed to mean “one or more” unless specified otherwise or clear from context to be directed to a singular form.

The cosmetic compositions and methods of the present invention can comprise, consist of, or consist essentially of the essential elements and limitations of the invention described herein, as well as any additional or optional ingredients, components, or limitations described herein or any otherwise useful ingredient found in personal care compositions intended for application to face scrubs, body scrubs, and other exfoliators.

“Antibacterial” as used herein refers to an activity that inhibits bacterial cells. In some embodiments, an antibacterial compound causes reduction in viability and/or growth of bacterial cells, e.g., the compound is toxic to bacterial cells. In some embodiments, an antibacterial acyl amino acid is an amino acid that, when contacted with bacterial cells, causes a reduction in the number and/or growth of the cells.

“Antimicrobial” as used herein refers to an activity that inhibits microbial cells. In some embodiments, an antimicrobial compound causes reduction in viability and/or growth of microbial cells, e.g., the compound is toxic to microbial cells.

Exfoliant

According to the present invention, the exemplary composition comprises at least one exfoliant or scrubbing agent, wherein said exfoliant comprises granular salt or fine granulated sugar. It is contemplated that the particle size of the granular salt or sugar is from 0.15 mm to 1.0 mm.

It is to be understood that the particle size of the salt or sugar is not so large so as to create irritation on the skin when scrubbed thereagainst. The salt or sugar may be present in the composition in a range from about 91.0% to 93.0% by weight of the total weight of the composition, including all ranges and subranges therebetween, and more preferably in a range from about 92.17% to 92.23% of the total weight of the composition.

Emollient

According to the present invention, the composition comprises at least one emollient, wherein said emollient is an oil such as soybean oil. Soybean oil provides a medium to adequately mix the composition. Additionally, soybean oil is used to moisturize and soothe skin, including from skin conditions such as blemishes and acne. One of the benefits of soybean oil is that it provides protection from UV rays to treat damages from the sung. Strong anti-oxidant properties of soybean oil also improve skin health and protect skin cells from free radical damage. Non-limiting examples of other suitable emollients include: safflower (Carthamus Tinctorius) oil, grape seed oil, avocado (Persea Gratissima) oil, jojoba (Simmondsia Chinensis) oil, and other types of oils.

According to preferred embodiments, the soybean oil is present in an amount sufficient to coat the exfoliant of the composition. It is to be understood that the amount of soybean oil needed to reduce dryness of the composition should depend upon the amount of exfoliant present in the composition. For example, the more exfoliant that is present, the more emollient should be added to reduce the dryness of the composition.

When present, care should be taken that the emollient is used at a level of no more than 8.0% by weight to avoid excessive fluid or liquid content. Generally speaking, preferred ranges of soybean oil in the composition is from about 6.0% to 8.0% by weight of the total weight of the composition, including all ranges and subranges therebetween, and more preferably in a range from about 7.07% to 7.08% of the total weight of the composition.

### Colorant

According to the present invention, the composition may comprise at least one coloring agent. The coloring agents having characteristics of being red, orange, yellow, green, blue, purple, pink, white, brown, and combinations of thereof may be used, depending upon embodiment. Additionally, other pigments such as nacreous pigments may be present in the composition. Non-limiting examples of other suitable colorants include: food coloring, organic pigments, FD&C dyes, D&C dyes, and/or a combination thereof.

It is to be understood that the amount of the colorant to be used depends upon the desired color. For example, the more colorant which is present, the more saturated the color of the composition. Generally speaking, preferred ranges of the colorant in the composition is from about 0.0% to 0.2% by weight of the total weight of the composition, including all ranges and subranges therebetween, and more preferably 0.12% of the total weight of the composition.

### Fragrance

According to the present invention, the composition may comprise at least one fragrance agent. Without limitation, fragrance having characteristics of being minty, floral, fruity, woody, sweet, citral, and combinations of thereof may be used, depending upon embodiment.

It is to be understood that the amount of fragrant used may depend upon the amount of scent desired. Generally speaking, preferred ranges of the fragrance agent in the composition is from about 0.1% to 0.3% by weight of the total weight of the composition, including all ranges and subranges therebetween, and more preferably 0.23% of the total weight of the composition.

### Vitamin/Tea Tree Oil

According to the present invention, the composition may comprise at least one vitamin E (tocopheryl acetate) component. Alternatively, the composition may comprise melaleuca alternifolia (tea tree) leaf oil or a combination of the vitamin E component and the tea tree oil. Both the tea tree oil and vitamin E are suitable for topical use as a remedy for a variety of skin disorders. In other embodiments, however, other vitamin compounds such as vitamin C (L-ascorbic acid), vitamin K (phytonadione), or a combination thereof may be used.

Generally speaking, preferred ranges of the vitamin E compound or the tea tree oil in the composition is from about 0.2% to 0.4% by weight of the total weight of the composition, including all ranges and subranges therebetween, and more preferably 0.35% of the total weight of the composition.

### Additional Ingredients

The composition of the present invention can also comprise any additive usually used in the field under consideration. In particular, other cosmetic and dermatological active agents such as, moisturizers, essential oils, vitamins, and mixtures thereof can be added. It is contemplated that the foregoing additional ingredients are those ordinarily used in the art, and are suitable for cosmetics use.

Namely, polysorbate 80 is used in embodiments where sugar is used instead of salt. Polysorbate 80 helps prolong the shelf life of the composition but does not affect the effectiveness of the composition. Generally speaking, preferred ranges of the polysorbate 80 in the composition is from about 0.0% to 0.1% by weight of the total weight of the composition, including all ranges and subranges therebetween, and more preferably 0.06% of the total weight of the composition.

### Example 1

In an exemplary embodiment, a body scrub composition was made according to the following formula in Table 1:

TABLE 1

| Ingredients | % (w/w) |
|---|---|
| Soybean Oil | 7.08 |
| Fragrance | 0.23 |
| Colorant | 0.12 |
| Vitamin E/Tea Tree Oil | 0.35 |
| Non-Iodized Fine Granular Salt | 92.23 |

In this regard, the non-iodized fine granular salt and the oil mixture are present in a weight ratio ranging from about 94:6 to about 92:8.

In another exemplary embodiment, a body scrub composition was made according to the following formula in Table 2:

TABLE 2

| Ingredients | % (w/w) |
|---|---|
| Soybean Oil | 7.08 |
| Fragrance | 0.23 |
| Colorant | 0.12 |
| Vitamin E/Tea Tree Oil | 0.35 |
| Non-Iodized Fine Granular Sugar | 92.17 |
| Polysorbate 80 | 0.06 |

In this regard, the non-iodized fine granular salt and the oil mixture are present in a weight ratio ranging from about 94:6 to about 92:8.

Process

1. 128 fl oz soybean oil, 4 fl oz fragrance, 2 fl oz colorant, and 2 fl oz vitamin E or tea tree oil were combined in a mixing container or barrel for approximately 10 minutes at room temperature, e.g., 21° C. to 22° C., to form an oil mixture. For the embodiment containing sugar, 1 fl oz polysorbate 80 was also added to the oil mixture.

2. The first one half 100 lbs or salt or 50 lbs of salt or sugar was added to the oil mixture and mixed until the first batch of the salt was well dispersed in the oil mixture, or until the mixture scraped the sides of the barrel.

3. The second half of 100 lbs of salt or 50 lbs of salt or sugar was added to the mixture and mixed until well blended and the mixture was uniform in composition.

4. After the composition was formed, it was packaged in a jar or a container.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. A face and body scrub composition, comprising:
6.0 to 7.0% by weight of composition soybean oil;
0.1 to 0.3% by weight of composition fragrance;
0.0 to 0.2% by weight of composition colorant;
0.2 to 0.4% by weight of composition tea tree oil;
91.0 to 93.0% by weight of composition exfoliant.

2. The scrub composition of claim 1, wherein said exfoliant comprises non-iodized fine granular salt.

3. The scrub composition of claim 1, wherein said exfoliant comprises sugar.

4. The scrub composition of claim 3, further comprising 0.0 to 0.06% by weight of composition polysorbate 80.

5. A face and body scrub composition, comprising:
6.0 to 7.0% by weight of composition soybean oil;
0.1 to 0.3% by weight of composition fragrance;
0.0 to 0.2% by weight of composition colorant;
0.2 to 0.4% by weight of composition vitamin E compound;
91.0 to 93.0% by weight of composition exfoliant.

6. The scrub composition of claim 5, wherein said exfoliant comprises non-iodized fine granular salt.

7. The scrub composition of claim 5, wherein said exfoliant comprises sugar.

8. The scrub composition of claim 5, further comprising 0.0 to 0.06% by weight of composition polysorbate 80.

9. A method of producing a face and body scrub composition, comprising:
combining 6.0 to 7.0% by weight of composition soybean oil, 0.1 to 0.3% by weight of composition fragrance, 0.0 to 0.2% by weight of composition colorant, and 0.2 to 0.4% by weight composition vitamin E compound or tea tree oil for ten minutes with constant mixing;
adding a first batch of exfoliant and mixing until blended;
adding a second batch of said exfoliant and mixing until blended.

10. The method of claim 9, wherein each of said first batch of exfoliant and said second batch of said exfoliant comprises 45 to 46% by weight of composition non-iodized fine granular salt.

11. The method of claim 9, wherein each of said first batch of exfoliant and said second batch of said exfoliant comprises 45 to 46% by weight of composition sugar.

12. The method of claim 9, further comprising the step of combining 0.0 to 0.06% by weight of composition polysorbate 80.
